SEQUENCE LISTING (23 sequences)

SEQ ID NO: 1 - DNA, 28 bp, Artificial sequence
  Ad1 oligonucleotide sequence
aagcagtggtatcaacgcagagtnnngg

SEQ ID NO: 2 - DNA, 29 bp, Artificial sequence
  Ad2 oligonucleotide sequence
aagcagtggtatcaacgcagagtnnnggg

SEQ ID NO: 3 - DNA, 28 bp, Artificial sequence
  Ad3 oligonucleotide sequence
aagcagtggtatcaacgcagagtggggg

SEQ ID NO: 4 - DNA, 29 bp, Artificial sequence
  Ad4 oligonucleotide sequence
aagcagtggtatcaacgcagagtnnsggg

SEQ ID NO: 5 - DNA, 27 bp, Artificial sequence
  Ad5 oligonucleotide sequence
aagcagtggtatcaacgcagagtnncc

SEQ ID NO: 6 - DNA, 30 bp, Artificial sequence
  Ad6 oligonucleotide sequence
aagcagtggtatcaacgcagagtnnsgggc

SEQ ID NO: 7 - DNA, 30 bp, Artificial sequence
  Ad7 oligonucleotide sequence
aagcagtggtatcaacgcagagtacggggg

SEQ ID NO: 8 - DNA, 30 bp, Artificial sequence
  SMART II Oligonucleotide
aagcagtggtaacaacgcagagtacgcggg

SEQ ID NO: 9 - DNA, 55 bp, Artificial sequence
  cDNA synthesis primer
aagcagtggtatcaacgcagagtactttttttttttttttttttttttttttttt

SEQ ID NO: 10 - DNA, 23 bp, Artificial sequence
  PCR primer
aagcagtggtatcaacgcagagt

SEQ ID NO: 11 - DNA, 32 bp, Artificial sequence
  beta actin gene specific primer
cgtcatactcctgcttgctgatccacatctgc

SEQ ID NO: 12 - DNA, 24 bp, Artificial sequence
  phospolipase A2 gene specific primer
cacctcaagatgaaaacagataac

SEQ ID NO: 13 - DNA, 27 bp, Artificial sequence
  transferrin receptor gene-specific primer
ttctcatggaagctatgggtatcacat

SEQ ID NO: 14 - DNA, 28 bp, Artificial sequence
  interferon- gamma receptor gene-specific primer
ggtgggggcttttattacggttatgagc

SEQ ID NO: 15 - DNA, 24 bp, Artificial sequence
  glyceraldehyde 3-phosphate dehydrogenase gene- specific primer
catgtgggccatgaggtccaccac

SEQ ID NO: 16 - DNA, 41 bp, Artificial sequence
  Ad8 oligonucleotide sequence
aagcagtggtatcaacgcagagtggccattacggccggggg

SEQ ID NO: 17 - DNA, 58 bp, Artificial sequence
  cDNA synthesis primer CDS-3M
aagcagtggtatcaacgcagagtggccgaggcggccttttttttttttttttttttvn

SEQ ID NO: 18 - DNA, 5 bp, Artificial sequence
  3′-end sequence of a deoxyribooligonucleotide adaptor
nnngg

SEQ ID NO: 19 - DNA, 6 bp, Artificial sequence
  3′-end sequence of a deoxyribooligonucleotide adaptor
nnnggg

SEQ ID NO: 20 - DNA, 5 bp, Artificial sequence
  3′-end sequence of a deoxyribooligonucleotide adaptor
ggggg

SEQ ID NO: 21 - DNA, 6 bp, Artificial sequence
  3′-end sequence of a deoxyribooligonucleotide adaptor
nnsggg

SEQ ID NO: 22 - DNA, 8 bp, Artificial sequence
  3′-end sequence of a deoxyribooligonucleotide adaptor
nnnsgggc

SEQ ID NO: 23 - DNA, 8 bp, Artificial sequence
  3′-end sequence of a deoxyribooligonucleotide adaptor
nnnsgggg

(19) United States
(12) Patent Application Publication
(10) Pub. No.: US 2008/0145844 A1
(43) Pub. Date: Jun. 19, 2008

(54) Methods of cDNA preparation

(75) Inventors: Ekaterina V. Barsova, Moscow (RU); Sergey A. Lukyanov, Moscow (RU)

Correspondence Address: PATTERSON & SHERIDAN, L.L.P., 3040 POST OAK BOULEVARD, SUITE 1500, HOUSTON, TX, 77056, US

(73) Assignee: Evrogen Joint Stock Company

(21) Appl. No.: 11/651,852

(22) Filed: Jan. 9, 2007

Related U.S. Application Data
(60) Provisional application No. 60/762,199, filed on Jan. 25, 2006.

(51) Int. Cl. C12Q 1/68; C12P 19/34

(52) U.S. Cl. 435/6; 435/91.2

(57) ABSTRACT

The present invention provides an improved method for cDNA preparation. The method of the present invention comprises the following steps: (1) contacting mRNA with a cDNA synthesis primer which can anneal to RNA and a suitable enzyme which possesses reverse transcriptase activity under conditions sufficient to permit the template-dependent extension of the primer to generate an mRNA-cDNA intermediates; (2) contacting a mixture from step 1 with a deoxyribonucleotide adapter in the presence of Mn²⁺-ions, wherein said oligonucleotide adapter has a pre-selected arbitrary nucleotide sequence at its 5′-end, and a short dG stretch at its 3′-end. The 3′-end nucleotide of the adapter is a terminator nucleotide, e.g., a nucleotide with a modified 3′-OH group of a deoxyribose residue.

### CROSS-REFERENCE TO RELATED APPLICATIONS

This application claims benefit of United States provisional patent application serial number 60/762,199, filed Jan. 25, 2006, which is herein incorporated by reference.

### FIELD OF THE INVENTION

The field of this invention is molecular biology, particularly improved technology for cDNA preparation.

### BACKGROUND OF THE INVENTION

ds cDNA synthesized on a template of poly(A)+RNA (mRNA) is widely used in various molecular biology applications as a physical resource for full-length clones. cDNA libraries constructed according to conventional methods (Wu, ed. Methods in Enzymology (1987), vol. 152) contain a high percentage of 5′-truncated clones due to the premature stop of reverse transcription (RT) of the template mRNA. In addition, there is a size bias against large fragments inherent in the cloning procedure. Therefore, large full-length cDNAs are strongly underrepresented in conventional libraries.

Several methods have been developed to construct cDNA libraries that are enriched in full-length cDNAs. These methods utilize either RNA oligo ligation to the 5′-end of mRNA (Kato et al., Gene 1994, V. 150, pp. 243-250; Suzuki et al., Gene 1997, v. 200, pp. 149-156), 5′-cap affinity selection via eukaryotic initiation factor 4E (Edery et al., Mol Cell Biol 1995, V. 15, pp. 3363-3371), or 5′-cap biotinylation followed by biotin affinity selection (Carninci and Hayashizaki, Methods Enzymol 1999, pp. 303:19-44; Carninci et al., Genomics 1996, V. 37, pp. 327-336). Common to these methods is that they are laborious, contain several enzymatic steps, are sensitive to quality loss through RNA degradation, and require high amounts of starting mRNA.

The SMART method seems most simple and requires only limited amounts (0.025-1 μg) of starting mRNA (U.S. Pat. No. 5,962,272; Zhu et al., Biotechniques 2001, V. 30, pp. 892-897; Wellenreuther et al., BMC Genomics 2004, V. 5(1); p. 36). The method utilizes properties of Moloney murine leukemia virus reverse transcriptases (MMLV RT e.g. SuperScriptII, Power Script and Expand-RT) to add a few non-template nucleotide residues (usually C) at the 3′-end of the first strand cDNA when they reach the end of the mRNA template. When added into a RT reaction, oligonucleotides containing an oligo(rG) at their 3′-end (template switching oligonucleotides) form pairs with the deoxycytidine stretch produced by MMLV-related transcriptase. The reverse transcriptase continues replication using the template switching oligonucleotide as a template, leading to 3′-addition of the template switching oligonucleotide complimentary sequence to serve as a universal 5′-terminal site for primer annealing during subsequent cDNA amplification.

The term “template switching oligonucleotide” refers to an oligonucleotide having a pre-selected arbitrary nucleotide sequence at its 5′-end and at least one ribonucleotide residue at its 3′-end. Possible structures and modifications of a template switching oligonucleotide are described in detail in U.S. Pat. No. 5,962,272. Ribonucleotide residues at 3′-end of the template switching oligonucleotide provides an effective template switching effect that is not practically observed when a deoxyribonucleotide adapter is used.

As used herein the term “template switching reaction” means a process of template-dependent synthesis of the complementary strand by a reverse transcriptase using two templates in consecutive order and which are not covalently linked to each other by phosphodiester bonds. The synthesized complementary strand will be a single continuous strand complementary to both templates. Usually, the first template is polyA+RNA and the second template is an oligonucleotide adapter.

As used herein, the term “arbitrary sequence” means any defined or pre-selected deoxyribonucleotide, ribonucleotide or mixed deoxyribo/ribonucleotide sequence.

As used herein, the term “mRNA:cDNA hybrid” refers to a product after first-strand cDNA synthesis catalyzed by reverse transcriptase using polyA+RNA as a template.

As used herein, the term “reverse transcriptase” or “RT” means any DNA polymerase possessing reverse transcriptase activity which can be used for first-strand cDNA synthesis using polyA+RNA or total RNA as a template.

As used herein, two sequences are said to be “complementary” to one another if they are capable of hybridizing to one another to form a double-stranded nucleic acid structure.

In spite of the availability of SMART technology, methods for constructing cDNA libraries enriched in full-length cDNA clones are desirable.

### SUMMARY OF THE INVENTION

The present invention provides an alternative method for cDNA preparation utilizing MMLV RT capacity to add a few non-template nucleotide residues at the 3′-end of the first strand cDNA when they reach the end of the mRNA template. The method of the present invention comprises the following steps: (1) annealing a cDNA synthesis primer to RNA template and synthesizing a first cDNA strand to form an RNA-cDNA intermediates (hybrids); (2) contacting a reaction mixture from step 1 with an oligonucleotide adapter in the presence of Mn²⁺-ions, wherein said oligonucleotide adapter is an oligonucleotide having a pre-selected arbitrary nucleotide sequence at its 5′-end, and a terminator deoxyribonucleotide at its 3′-end. In preferred embodiments, the oligonucleotide adapter comprises two or more dG at its 3′-end.

In certain embodiments, steps 1 and 2 of the method are separated in time. In a preferred embodiment, step 1 is directly followed by step 2. In some embodiments, steps 1 and 2 of the method are performed simultaneously.

The resulting ss cDNA includes the 5′-end of the RNA molecule as well as the sequence complementary to the adapter, which can then serve as an universal priming-site in subsequent amplification of the cDNA.

Deoxyribonucleotide adapters that can be used according to the subject method and kits containing the deoxyribonucleotide adapter are also included within the scope of the present invention.

### BRIEF DESCRIPTION OF THE DRAWINGS

FIG. 1 provides a schematic outline of a cDNA synthesis method, according to an embodiment of the invention.

FIG. 2 provides an inverted photo of PCR amplification products revealed using agarose gel electrophoresis with ethidium bromide staining. First strand cDNA synthesis was performed with SMART II Oligonucleotide (lane 1); Ad1 (lane 2) or Ad1P (lane 3). M—1 kb DNA size markers (SibEnzyme).

FIG. 3 provides results of PCR amplification of the first strand cDNA produced using Ad1P adapter in the presence of different divalent cations: Lanes 1-4, Co²⁺ (from left to right: CoSO₄ with final concentration 3 mM, 1.5 mM, 0.75 mM, 0.37 mM); Lanes 5-8, Mn²⁺ (from left to right: MnCl₂ with final concentration 3 mM, 1.5 mM, 0.75 mM, 0.37 mM); Lanes 9-13, Ca²⁺ (from left to right: CaCl₂ with final concentration 15 mM, 7.5 mM, 3.7 mM, 1.9 mM, 0.9 mM). M —1 kb DNA size markers (SibEnzyme).

FIG. 4 provides results of PCR amplification of the first strand cDNA produced in the presence of Mn²⁺-ions with different deoxyribonucleotide adapters with (lanes 1-3) or without (lanes 4-6) cDNA synthesis primer. Adapters are SMART II Oligonucleotide (lanes 1, 4); Ad1 (lanes 2, 5) and Ad1P (lanes 3, 6). M —1 kb DNA size markers (SibEnzyme).

FIG. 5 provides results of PCR amplification of the first strand cDNA produced in the presence of Mn²⁺-ions with SMART II Oligonucleotide (lanes 2, 13), adapter Ad1 (lane 3), Ad1P (lane 4), Ad2ddG (lane 5), Ad2acG (lane 6), Ad4NH2 (lane 7), Ad6ddC (lane 8), Ad4P (lane 9), Ad1P (lane 10), Ad5P (lane 11), Ad3 (lane 12). SMART cDNA prepared by standard SMART protocol is shown in lane 1. M—1 kb DNA size markers (SibEnzyme).

FIG. 6 provides results of PCR amplification of the first strand cDNA produced by two steps first-strand cDNA synthesis with different times of template switching reaction: lane 1-0 min; lane 2-1 min.; lane 3-5 min; lane 4-15 min; lane 5-30 min; lanes 6, 9-50 min; lane 7-60 min; lanes 8, 10-90 min; lane 11-120 min. Ad1P adapter was used.

FIG. 7 provides results of PCR amplification of the first strand cDNA produced by two steps first-strand cDNA synthesis with the Ad7P adapter. M —1 kb DNA size markers (SibEnzyme).

FIG. 8 provides results of PCR amplification of the first strand cDNA produced by two steps first-strand cDNA synthesis with the Ad8P adapter. M —1 kb DNA size markers (SibEnzyme).

### DETAILED DESCRIPTION OF THE INVENTION

As summarized above, the present invention is directed to a composition and method to synthesize cDNA from total or poly A+ RNA. The method of the present invention is primarily based on the ability of some reverse transcriptases (e.g., MMLV-related reverse transcriptases) to add a few non-template nucleotide residues (usually deoxycytidine) at the 3′-end of the first strand cDNA when they reach the end of the mRNA template.

As used herein, the term “MMLV-related reverse transcriptase” or “MMLV RT” refers to a wild type Moloney murine leukemia virus reverse transcriptase and it variants, for example mutants lacking RNase H activity such as SuperScriptII, Power Script and Expand-RT.

It has been previously discovered that only template switching oligonucleotides having at least one ribonucleotide residue (usually 3-5 riboG) at its 3′-end can be used as an effective template for template switching reaction (U.S. Pat. No. 5,962,272; Zhu et al., Biotechniques 2001, V. 30, pp. 892-897; Wellenreuther et al., BMC Genomics 2004, V. 5(1); p. 36). Deoxyribonucleotide oligonucleotides including those with blocked 3′-ends were found ineffective for template switching reactions.

The inventors have discovered that an oligonucleotide adapter that does not include ribonucleotide residues at its 3′-end can be used as an effective template for a template switching reaction when the template switching reaction is performed in the presence of Mn²⁺-ions. Therefore, the present invention is directed to a composition and method to synthesize cDNA from total or poly A+ RNA using an oligonucleotide adapter that does not include ribonucleotide residues at its 3′-end.

The method of the present invention comprises the following steps: (1) annealing a cDNA synthesis primer to RNA template and synthesizing a first cDNA strand to form an RNA-cDNA intermediates (hybrids); (2) contacting a reaction mixture from step 1 with an oligonucleotide adapter in the presence of Mn²⁺-ions, wherein said oligonucleotide adapter is an oligonucleotide having a pre-selected arbitrary nucleotide sequence at its 5′-end, and a terminator deoxyribonucleotide at its 3′-end. In preferred embodiments, the oligonucleotide adapter comprises two or more dG at its 3′-end.

At step 2, first strand cDNA molecules synthesized at step 1 are elongated by reverse transcriptase using the oligonucleotide adapter of the present invention as a template. The resulting ss cDNA includes the complete 5′-end of the RNA molecule as well as the sequence complementary to the adapter, which can then serve as a universal priming site in subsequent cDNA amplification.

In certain embodiments, steps 1 and 2 of the method are separated in time. In a preferred embodiment, step 1 is directly followed by step 2. In some embodiments, steps 1 and 2 of the method are performed simultaneously.

Adapter Structure

In certain embodiments, the adapter of the present invention is a chemically synthesized oligonucleotide. In certain embodiments, the adapter of the present invention comprises deoxyribonucleotides selected from the group consisting of dAMP, dCMP, dGMP and dTMP. In certain embodiments, the 3′-end residue of the adapter is a terminator nucleotide or nucleotide analog (e.g. 3′-amino NMP, 3′-phosphate NMP, 3′-fluoro NMP, dideoxyribonucleotide, or acyclonucleotide) that prevents elongation of the adapter by reverse transcriptases. In preferred embodiments, this nucleotide residue is a purine, more preferably, a guanosine or guanosine analog. In all embodiments, the oligonucleotide adapter of the present invention does not contain a ribonucleotide at its 3′-end. In preferred embodiments, the 3′-terminal guanosine has a modified 3′-OH group of deoxyribose residue by an amino, biotin, phosphate, fluoro or glycerol group, dideoxyribonucleotide, or acyclonucleotide.

As used herein, the term “nucleotide analog” means a nucleotide which is not typically found in DNA or RNA. Nucleotide analogs include modifications in the base or sugar-phosphate backbone. For example, nucleotide analogs include peptide nucleic acids, inosine, 5-nitroindole deoxyribofuranosyl, 5-methyldeoxycytosine, and 5,6-dihydro-5,6-dihydroxydeoxythymidine. Other nucleotide analogs will be evident to those skilled in the art.

In many embodiments, an adapter of the present invention is at least 12 bp long, in certain embodiments at least 15 bp long, usually 15-50 bp long, and more usually 20-35 bp long.

In certain embodiments, an oligonucleotide adapter of the present invention has 2 or more guanosine residues at its 3′-end (usually a 3-7 nucleotide stretch). In certain embodiments, said adapter comprises a pre-selected arbitrary nucleotide sequence at its 5′-end. The arbitrary sequence can be selected so as to be useful for subsequent cDNA synthesis and cloning steps.

In certain embodiments, an oligonucleotide adapter of the present invention also comprises 2 or 3 random nucleotide residues near to its 3′-end, before the dG stretch. In certain embodiments, the 3′-end of the adaptor has a structure selected from the group consisting of NNNGmG (SEQ ID NO: 18), NNNGGmG (SEQ ID NO: 19), GGGGmG (SEQ ID NO: 20), NNSGGmG (SEQ ID NO: 21), NNNSGGGmC (SEQ ID NO: 22), NNNSGGGmG (SEQ ID NO: 23), wherein N is a random deoxyribonucleotide, S is dC or dG, mG or mC are terminator deoxyribonucleotides, e.g., nucleotides having a modified 3′-OH group of deoxyribose residue by an amino, biotin, phosphate, fluoro or glycerol group or a dideoxiribonucleotide or an acyclonucleotide.

As used herein, the term “terminator deoxyribonucleotide” or “terminator nucleotide” refers to a deoxyribonucleotide or deoxyribonucleotide analog with a modified 3′-OH group. When inserted into the 3′-end of an oligonucleotide, it prevents the oligonucleotide from being used as a primer in the DNA elongation or PCR reactions.

In certain embodiments, an oligonucleotide adapter of the present invention can interact with intermediates of reverse transcriptase-mRNA-cDNA which are generated at the 5′-end of full-length mRNA after first-strand cDNA synthesis, wherein said cDNA in the intermediate contains a few non-template nucleotide residues (usually C) at the 3′-end. In certain embodiments, an oligonucleotide adapter of the present invention serves as a template for elongation of cDNA by reverse transcriptase in the presence of Mn²⁺-ions.

An adapter of the present invention can be modified by any methods known in the art. For example, the adapter can be modified to provide an advantage for selective binding to the CAP structure of mRNA, for example by covalent binding of the adapter with a protein capable of binding the CAP structure of mRNA (see U.S. Pat. No. 5,219,984), or with a CAP-specific aptamer (Kenan et al., Trends Biochem. Sci. 1994, V. 19, pp. 57-64). An adapter linked to a polypeptide capable of recognizing and binding the CAP structure (e.g. initiation factor 4E protein) can be also prepared. Adapter structure can be also modified using well known approaches, such as replacement of deoxyribonucleotides with random nucleotides, nucleotide analogs, or nucleotides labeled with different hapten groups (such as, for example, biotin, digoxigenin, and fluorescein), using partially double-stranded DNA containing an extension of a single-stranded adapter sequence, incorporation of restriction sites, etc.

As used herein, the term “hapten” means a molecule that can bind to a nucleic acid molecule and can be recognized and bound by another molecule, or “binding ligand,” e.g., an antibody, streptavidin and biotin, transient metal, and others known in the art. Examples of haptens include chelating groups, biotin, fluorescein, digoxigenin, antigens, and others known in the art.

As used herein, the term “random sequence” means a deoxyribonucleotide, ribonucleotide or mixed deoxyribo/ribonucleotide sequence which contains in each nucleotide position any natural or modified nucleotide.

Step 1 of the First-Strand cDNA Synthesis.

The first step of the method of the present invention is a conventional first-strand cDNA synthesis. As a starting material for cDNA synthesis, poly(A)+RNA or total RNA can be used. First-strand cDNA synthesis is carried out by reverse transcriptase, or by a DNA polymerase possessing reverse transcriptase activity under suitable conditions. A cDNA synthesis primer is used to initiate cDNA synthesis.

Suitable conditions for synthesizing first cDNA strands using RNA as templates include the presence of effective amounts of a reverse transcriptase and other reagents, such as buffers and a deoxynucleotide triphosphate mixture (dNTPs), at appropriate temperatures and for sufficient lengths of time.

At first, a cDNA synthesis primer is annealed to the template mRNA. For example, 10 pmol of the primer is contacted with an appropriate amount of RNA (usually from 0.05 to 2 μM) in a volume of 5 μl of sterile water, the mixture is heated for 2 minutes at 70° C. Then the mixture is contacted with other components of cDNA synthesis reaction, e.g. with appropriate amounts of deoxyribonucleoside triphosphates (ATP, CTP, GTP and TTP), Mg²⁺-ions, an optimal buffer for reverse transcriptase and the reverse transcriptase enzyme. For example the RNA-primer complex is contacted with the reverse transcriptase (e.g. 1 μl of SuperScript) in a final volume of 10 μl, containing 50 mM Tris-HCl (pH 8.3 at 22° C.); 75 mM KCl; 6 mM MgCl₂; 1 mM DTT; and 1 mM each of dATP, dGTP, dCTP, and dTTP. cDNA synthesis reaction is performed at 42° C. for at least 20 min, usually at least 40 min, more usually at least 60 min, and often for 1.5-2 hrs.

A variety of enzymes possessing reverse transcriptase activity can be used for the first-strand cDNA synthesis. In the scope of the invention are enzymes that add a few non-template nucleotide residues (e.g., deoxycytidine) at the 3′-end of the first strand cDNA when they reach the end of the mRNA template. Examples of suitable enzymes include but are not limited to the reverse transcriptase from Moloney murine leukemia virus (MMLV), which is described in U.S. Pat. No. 4,943,531, which is herein incorporated by reference, and MMLV reverse transcriptase lacking RNaseH activity, which is described in U.S. Pat. No. 5,405,776, which is herein incorporated by reference, or any variant of MMLV reverse transcriptase.

In a preferred embodiment, the first-strand cDNA synthesis reaction does not contain Mn²⁺-ions and an oligonucleotide adapter. In some embodiments, step 1 of the first-strand cDNA synthesis reaction can include Mn²⁺-ions or an oligonucleotide adapter or both components.

The cDNA synthesis primer structure may be varied depending on the strategy to be employed for cDNA cloning. The primer can be a single-stranded (ss) or a double-stranded (ds) oligonucleotide, or combination of ss and ds portions (e.g. primer-restriction-end adapter, as described by Coleclough et al., Gene, 1985, V. 34, pp. 305-314). In certain embodiments, the cDNA synthesis primer is a modified oligo(dT) primer, e.g., a primer comprising a (dT)-stretch capable of annealing to the poly(A) tail of mRNA. In some embodiments, a random primer or a gene-specific primer can also be used to initiate cDNA synthesis. In certain embodiments, the 5′-end sequence of the cDNA synthesis primer comprises a pre-selected arbitrary nucleotide sequence. In some embodiments, this sequence is at least in part complementary to the 5′-sequence of the oligonucleotide adapter used at the step 2 of the method.

Step 2 of the First Strand cDNA Synthesis.

At step 2 of the method the mRNA-cDNA-reverse transcriptase intermediates are contacted with an oligonucleotide adapter of the present invention described above under conditions ensuring effective template switching reaction, i.e., in the reverse transcriptase buffer containing Mn²⁺-ions.

The concentration of Mn²⁺-ions used varies depending on the particular reverse transcriptase that is employed. However, in many embodiments, concentration of Mn²⁺-ions is from about 0.1 to about 10 mM, usually from about 0.3 to about 7 mM, more usually from about 0.5 to about 6 mM, e.g. from about 1 to about 5 mM.

The concentration of the oligonucleotide adapter varies depending on the particular adapter structure and reaction conditions that are employed. However, in many embodiments, concentration of an oligonucleotide adapter is from about 0.5 to about 4 μM, more usually from about 1 to about 3 μM, and more usually from about 1.6 to about 2.1 μM.

Suitable conditions for the template switching reaction include the presence of a cDNA synthesis reaction from step 1 and other reagents, such as Mn²⁺-ions and a deoxynucleotide adapter, at appropriate temperatures and for sufficient lengths of time. Usually the reaction is performed at 42° C. for a least 20 min, usually at least 40 min, more usually at least 60 min, and often for 1.5-2.5 hrs.

Use

The method of the present invention can be used to synthesize ss cDNA on a template of poly(A)+ or total RNA. cDNA obtained by this method will be flanked by pre-selected arbitrary sequences and can be applied for wide range of molecular biology applications. For example, said ss cDNA can be used as a template in RACE (rapid amplification of cDNA ends) approach. For example, this ss cDNA can be used for RACE described in Matz et al., Nucleic Acids Res. 1999, V. 27(6), pp. 1558-1560, and Matz et al., Methods Mol. Biol. 2003, V. 221, pp. 41-49. In particular, to isolate the 5′-end of the cDNA of interest, ss cDNA obtained by the method of the present invention can be contacted with an oligonucleotide primer corresponding partially or completely to the nucleotide sequence of the adapter of the present invention, a gene-specific primer corresponding to the known sequence of the cDNA of interest, and an effective amount of other reagents essential to perform polymerase chain reaction (PCR), and subjected to PCR cycling to generate amplification product corresponding to the 5′-end fragment of a target cDNA.

In some embodiments, said ss cDNA can be inserted into recombinant cloning vehicles, and hosts can be transformed with these vehicles according to conventional methods well known in art (Kimmel and Berger, Meth. Enzymol. 1987, V. 152, pp. 307-316).

Said ss cDNA can be also used as a template to produce representative ds cDNA suitable for subsequent cloning using conventional procedures. The methods to produce ds cDNA from ss cDNA are well known in the art and can include:

1. Amplification of ds cDNA using known flanking sequences as sites for primer annealing. In this embodiment, ss cDNA obtained by the method of the present invention is contacted with an oligonucleotide primer comprising a portion of the nucleotide sequence of the adapter of the present invention, a primer partially or completely corresponding to the cDNA synthesis primer (previously used in the first strand synthesis procedure), and an effective amount of other reagents essential to perform PCR; and subjected to PCR cycling to generate an amplification product corresponding to the representative ds cDNA. In a preferred embodiment, PCR conditions allow amplification of long nucleic acid sequences, e.g., as described by Barnes (U.S. Pat. No. 5,436,149).

2. Second strand cDNA synthesis as described for example by Gubler and Hoffmanm (Gene 1983, V. 25, pp. 253-269). This process includes digestion of the RNA with a ribonuclease such as E. coli RNase H, repair synthesis using a DNA polymerase having the activities of DNA polymerase I, and ligation. The procedure depends on the structure of the cDNA synthesis primer used for the first-strand cDNA synthesis.

Representative ds cDNA can be used for cloning and preparation of representative cDNA library, by the methods well known in the art, as a starting material for cDNA subtractive hybridization as described for example in U.S. Pat. No. 5,565,340; U.S. Pat. No. 5,436,142; Hampson et al., Nucl. Acids Res. 1992, V. 20, pp. 2899; Yang et al., Anal. Biochem., 1996, V. 237, pp. 109-114; Balzer et al., Nucl. Acids Res. 1994, V. 22, pp. 2853-2854, for cDNA normalization, as described in Zhulidov et al., Nucleic Acids Res. 2004, V. 32(3), p.e37, and for other applications known in the art, including Virtual Northern blots (Franz et al., Nucleic Acids Res., 1999, V. 27, p. e3.), as a hybridization probe (see e.g. U.S. Pat. No. 5,484,702; U.S. Pat. No. 5,500,356; U.S. Pat. No. 4,888,274), and for specific removal of known sequences (cDNA depletion), etc.

Kits

Also provided by the present invention are kits for use in practicing one or more of the above-described applications. Kits typically include the oligonucleotides necessary for steps 1 and 2 of first strand cDNA synthesis and Mn²⁺-ions-containing solution to perform step 2 of first strand cDNA synthesis. In addition to the above components, the subject kits will further include instructions for practicing the subject methods. These instructions may be present in the subject kits in a variety of forms, one or more of which may be present in the kit.

The following example is offered by way of illustration and not by way of limitation.

### EXAMPLES

### Example 1

### Testing of the Deoxyribonucleotide Adapters

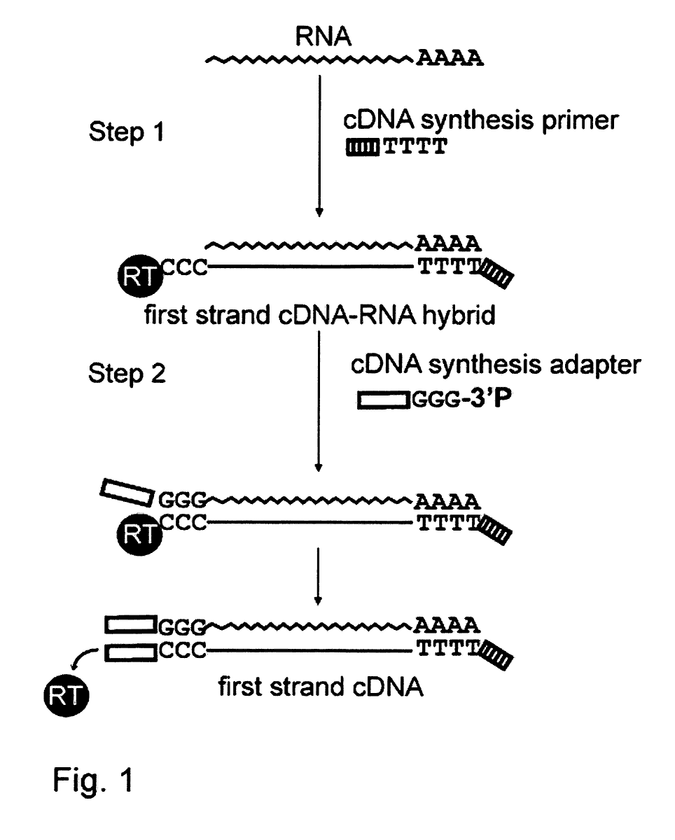

A schematic diagram of the cDNA synthesis method is provided in FIG. 1. The general distinction of the method provided from the previously developed template switching based approach described in U.S. Pat. No. 5,962,272 is use of the entirely deoxyribonucleotide adapter instead of a template switching oligonucleotide. It has been previously shown that under standard conditions, a deoxyribonucleotide oligonucleotide is noticeably less effective for template switching reaction than a template switching oligonucleotide having at least one ribonucleotide residue at its 3′-end.

The inventors have discovered that an oligonucleotide adapter that does not include ribonucleotide residues at its 3′-end can be used as an effective template for a template switching reaction when the template switching reaction is performed in the presence of Mn²⁺-ions. The following experiments were performed to test the effectiveness of the deoxyribonucleotide adapters for a template switching reaction in the presence of Mn²⁺-ions.

In the first experiment, template switching reactions were performed using a SMART II Oligonucleotide (Clontech) that has three riboguanosine resides at its 3′ end and is a template switching oligonucleotide described in U.S. Pat. No. 5,962,272 (SEQ ID NO: 8) and two deoxyribonucleotide adapters having the nucleotide sequence shown in SEQ ID NO: 1, wherein the first adapter (Ad1) has an unmodified nucleotide residue at the 3′-end, while the second (Ad1P) comprises a modified nucleotide residue with a 3′-phosphate group. Total RNA (Human Cerebellum) was used as a template for cDNA synthesis. The reaction was performed durante absentia Mn²⁺-ions under standard conditions described in the manufacturer's instruction of the SMART PCR cDNA Synthesis Kit (Clontech), which is herein incorporated by reference.

10 pmol of cDNA synthesis primer (SEQ ID NO: 9) and 10 pmol of the adapter (or SMART II Oligonucleotide) were annealed to 1 μg of total RNA in a volume of 5 μl of sterile water by heating the mixture for 2 minutes at 70° C., followed by cooling on ice for 2 min. First-strand cDNA synthesis was then initiated by mixing the primer-RNA mixture with 1 μl of Power Script reverse transcriptase (Clontech) in a final volume of 10 μl, containing 50 mM Tris-HCl (pH 8.3); 75 mM KCl; 6 mM MgCl₂; 1 mM DTT; and 1 mM each of dNTP. The first-strand cDNA synthesis-template switching reaction was incubated at 42° C. for 1.5 hours in an air incubator and then cooled on ice.

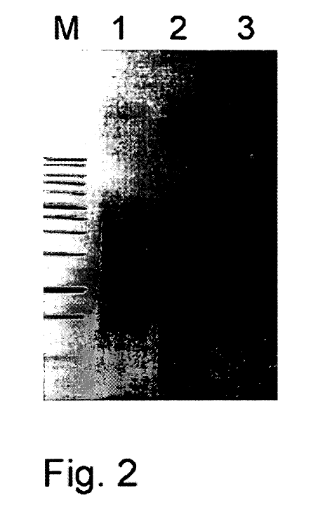

On the reaction completion, first strand cDNAs synthesized were diluted 5 times and 1 μl aliquots of the dilution were used for PCR amplification using the Advantage 2 PCR Kit (Clontech) with the PCR primer (SEQ ID NO: 10). 20 PCR cycles (95° C.-20 sec; 66° C.-20 sec; 72° C.-3 min) were performed. PCR products were revealed by agarose gel electrophoresis (FIG. 2). It was demonstrated that the Ad1P adapter does not stand as a template switching oligonucleotide under standard conditions. It was also demonstrated that Ad1 adapter is essentially less effective for a template switching reaction than a template switching oligonucleotide. These data confirm the results described in the U.S. Pat. No. 5,962,272.

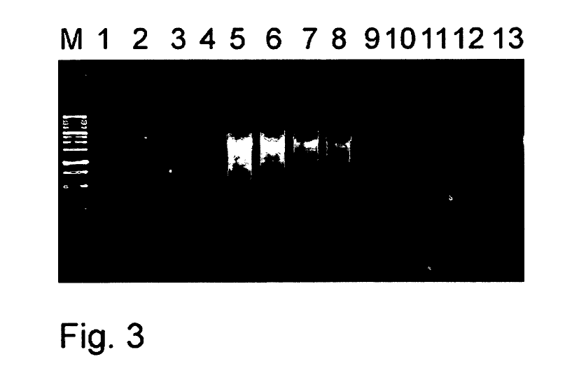

In the second experiment, the influence of the divalent cations on the template switching reaction with Ad1P was tested. Just as in the first experiment, first-strand cDNA synthesis (step 1) and the template switching reaction (step 2) were performed at one time under conditions described above with the one exception: different concentrations of divalent cations (Ca²⁺; Mn²⁺; or Co²⁺) were added into the reaction mixtures. Products of the first strand cDNA synthesis reaction were used for PCR amplification as described above and revealed on an agarose gel (FIG. 3). It was found that Mn²⁺-ions essentially increase cDNA yield in the case of Ad1P adapter, denoting that this adapter becomes effective for template switching reaction in the presence of Mn²⁺-ions. No substantial effects on the cDNA synthesis were observed in the cases of Ca²⁺ and Co²⁺-ions.

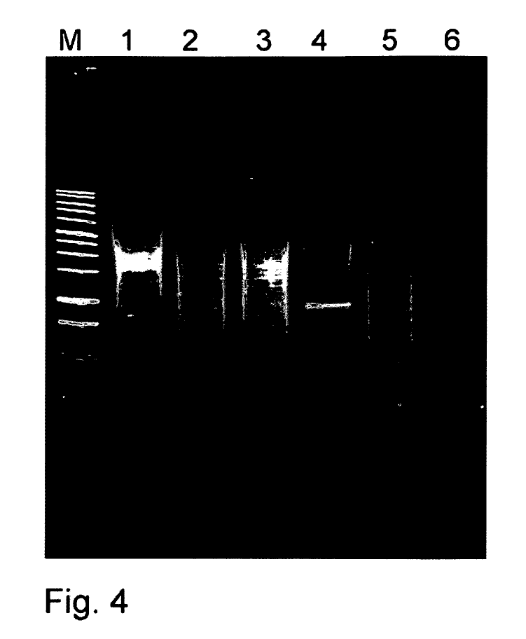

The third experiment reveals the contribution of the non-specific amplification from different adapters in the cDNA synthesis result. The cDNA synthesis reactions described in the first experiment were repeated in the presence of 3 mM MnCl₂ with or without cDNA synthesis primer. PCR products were revealed by agarose gel electrophoresis (FIG. 4). It was demonstrated that Ad1P adapter produces a smaller background as compared with Ad1 adapter and SMART II Oligonucleotide.

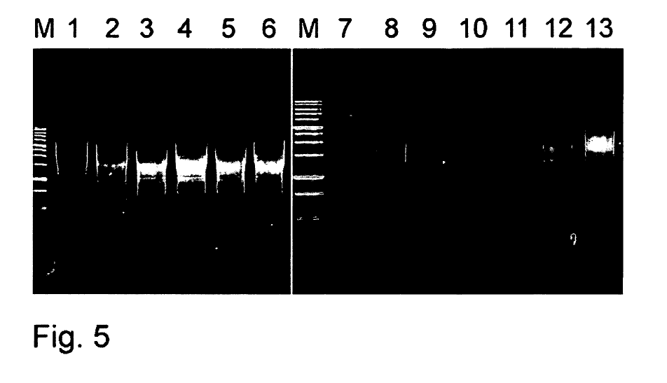

In the fourth experiment, different deoxynucleotide adapters were tested for suitability for a template switching reaction including Ad1, Ad1P, Ad2ddG (adapter structure is shown in SEQ ID NO: 2, wherein the 3′-nucleotide residue is dideoxyribonucleotide), Ad2acyG (adapter structure is shown in SEQ ID NO: 2, wherein the 3′-nucleotide residue is acycloguanin), Ad3 (adapter structure is shown in SEQ ID NO: 3), Ad4P (adapter structure is shown in SEQ ID NO: 4, wherein the 3′-end nucleotide residue comprises a 3′-phosphate group), Ad5P (adapter structure is shown in SEQ ID NO: 5, wherein the 3′-end nucleotide residue comprises a 3′-phosphate group), Ad4NH2 (adapter structure is shown in SEQ ID NO: 4, wherein the 3′-end nucleotide residue comprises a 3′-aminogroup), Ad6ddC (adapter structure is shown in SEQ ID NO: 6, wherein the 3′-nucleotide residue is dideoxyribocytosine). SMART II Oligonucleotide was used as a control. cDNA synthesis was performed in the presence of 3 mM MnCl₂, with or without a cDNA synthesis primer. It was found that few or no PCR products are generated from dideoxynucleotide adapters in the absence of a cDNA synthesis primer. In the presence of the cDNA synthesis primer and MnCl₂, Ad5P was found ineffective, whereas Ad4P, Ad4ddC, Ad1P, Ad2ddG, Ad6ddC, and Ad2acyG were found to be effective adapters for cDNA synthesis accompanying a template switching reaction (FIG. 5).

### Example 2

### cDNA Synthesis Optimization

The inventors have found that a deoxyribonucleotide adapter with a modified 3′-end can be utilized by reverse transcriptase as a second template in a template switching reaction only in the presence of Mn²⁺-ions. This indicates that Mn²⁺-ions influence reverse transcriptase substrate specificity. There are several reports suggesting that Mn²⁺-ions alter substrate specificity of some reverse transcriptases (e.g., Marcus and Modak, Nucleic Acid Res. 1976, V. 3, pp. 1473-1486; Vartanian et al., Journal of General Virology 1999, V. 80, pp. 1983-1986) and may increase misincorporations of dNTPs during first strand cDNA synthesis. To prevent these, the first and the second steps of the method were separated in time.

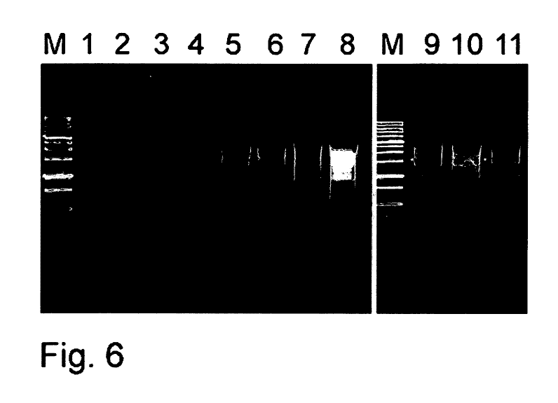

10 pmol of cDNA synthesis primer (SEQ ID NO: 9) was mixed with 500 ng of total RNA (Human Cerebellum) in a volume of 5 μl of sterile water and annealed to RNA by heating the mixture for 2 minutes at 70° C., followed by decreasing the temperature to 42° C. First-strand cDNA synthesis was then initiated by mixing the primer-RNA mixture with 1 μl of Power Script reverse transcriptase (Clontech) in a final volume of 10 μl, containing 50 mM Tris-HCl (pH 8.3); 75 mM KCl; 6 mM MgCl₂; 1 mM DTT; and 1 mM each of dNTP. The first-strand cDNA synthesis reaction was incubated at 42° C. for 1 hour under mineral oil. After this, the template switching reaction was initiated by addition of MnCl₂ (to final concentration 3 mM) and 10 pmol of the Ad1P adapter to the RNA-first strand cDNA-revertase intermediates. Incubation was prolonged for 2 hours. Aliquots were collected after 0, 1, 5, 15, 30, 50, 60, 90, and 120 min after Ad1P adapter and MnCl₂ addition and used for PCR with the PCR primer (SEQ ID NO: 10) as described in Example 1. PCR products were revealed by gel electrophoresis on 1.2% agarose with ethidium bromide (FIG. 6). It was found that 90 min is an optimal time for the template switching reaction.

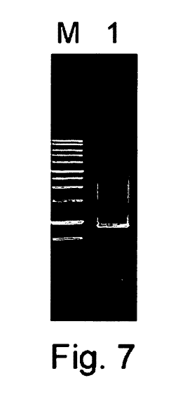

A further 10 pmol of the cDNA synthesis primer (SEQ ID NO: 9) was mixed with 500 ng of polyA RNA (Human Placenta) in a volume of 5 μl of sterile water and annealed to RNA by heating the mixture for 2 minutes at 70° C., followed by decreasing the temperature to 42° C. The reaction mix was covered by mineral oil. First-strand cDNA synthesis was then initiated by mixing the primer-RNA mixture with 1 μl of Power Script Reverse Transcriptase (Clontech) in a final volume of 10 μl, containing 50 mM Tris-HCl (pH 8.3); 75 mM KCl; 6 mM MgCl₂; 1 mM DTT; and 1 mM each of dNTP. The first-strand cDNA synthesis reaction was incubated at 42° C. for 40 minutes under mineral oil. After this, the template switching reaction was initiated by addition of a 5 μl mixture of the following components: 1 μl of 5× Buffer (250 mM Tris-HCl (pH 8.3); 375 mM KCl; 30 mM MgCl2), 2 μl of 22 mM MnCl₂ (to final concentration 3 mM) and 2 μl of the Ad7P adapter (24 pmol, adapter structure is shown in SEQ ID NO: 7, wherein the 3′-end nucleotide residue comprises a 3′-phosphate group) to the RNA-first strand cDNA-revertase intermediates. Incubation was prolonged for 90 min after Ad7P adapter and MnCl₂ addition. A 0.5 μl aliquot was used for 17 PCR cycles with the PCR primer (SEQ ID NO: 10) as described in Example 1. PCR products were revealed by gel electrophoresis on 1.2% agarose with ethidium bromide (FIG. 7).

### Example 3

### Cloning of 5′-End Sequences of Full-Length cDNA

Isolation of a full-length cDNA is an important and often one of the most difficult tasks in gene characterization. The method of the present invention allows synthesis of cDNA highly suitable for 5′-RACE procedure. First strand cDNA as well as amplified cDNA prepared on its base can be used for 5′-RACE by the different methods including Step-Out RACE procedure described in Matz et al., Nucleic Acids Res. 1999, V. 27(6), pp. 1558-1560, and Matz et al., Methods Mol. Biol. 2003, V. 221, pp. 41-49. ds cDNAs were prepared on the base of 0.5 μg of total RNA from Human HeLa cell as described in the Example 2 using Ad2P, Ad6ddC, Ad4P, Ad3P and Ad7P oligonucleotide adapters. cDNA samples were used for 5′-RACE with specific primers for human genes: beta actin (SEQ ID NO: 11), phospholipase A2 (SEQ ID NO:12), transferrin receptor (SEQ ID NO:13), interferon-gamma receptor (SEQ ID NO:14), and glyceraldehyde 3-phosphate dehydrogenase (SEQ ID NO:15);. 5′-RACE was performed as described in Matz et al., Nucleic Acids Res. 1999, V. 27(6), pp. 1558-1560. PCR products were analysed on an agarose gel. No remarkable difference was found for all cDNA samples tested. In each case, 5′-RACE reactions generated bands corresponding to the expected size of full-length amplified 5′-RACE products. Subsequent cloning and sequence analysis of 6 randomly picked 5′-RACE clones confirmed their identity to target gene 5′-end fragments.

### Example 4

### Preparation of cDNA Libraries

The method of the present invention allows synthesis of cDNA that can be effectively used for construction of cDNA libraries of different types (e.g. standard, normalized, subtracted, depleted) using as a template from 100-1000 ng of total or poly(A)+RNA. Any conventional procedure well known in art can be used to purify this small amount of total RNA from cells or tissues. It will be apparent to those skilled in the art that some individual non-essential steps, the structure of the cDNA synthesis primer, and adaptors can be varied without changing the efficiency of the procedure. For example, many conventional procedures well known in the art for cloning of PCR products, such as TA-cloning vectors, blunt end ligation, and the like, can be used for cloning and generation of cDNA libraries. Any such variations in the preferred protocol which are based on using methods and materials of the subject invention are within the scope of the invention.

1 μg of total RNA (Human Skeletal Muscle) was used for preparation of amplified cDNA as described in Example 2 using the Ad7P adapter. An aliquot of the cDNA prepared was used for cDNA normalization performed using a Trimmer cDNA Normalization Kit (Evrogen) according to the manufacturer's instructions. Non-normalized and normalized amplified cDNA were size fractionated using 0.5-5 kb, cloned into BlueScript 2KS+ vector, and transformed into E. coli strain XL 1 Blue. In order to confirm the high quality of the library generated, 94 white colonies from each library were randomly picked and insert sizes were determined for each clone. The size distribution of the inserts was in the range of 0.5-5.0 kb with a maximum of 1.5-3.0 kb that corresponds to the size distribution of skeletal muscle in total RNA used for cDNA library construction. An aliquot of the cDNA prepared was also used for suppression subtractive hybridization using a PCR-Select Kit (Clontech) according to the manufacturer's instructions. Human lung cDNA prepared exactly as described above was used as a driver. These data demonstrates that method of the present invention provides cDNA suitable for cDNA library construction and cDNA normalization and subtraction.

An expression library to identify genes encoding fluorescent proteins was also prepared. Total RNA was purified from Zoanthus sp. using TRIzol Reagent (Life Technologies). 0.5 μg of the total RNA was used for first strand cDNA synthesis and a template switching reaction as described in Example 2. The Ad7P adapter was used. After first strand cDNA synthesis and template switching reaction completion, cDNA was amplified as described in Example 2. 20 PCR cycles were performed. Amplified cDNA was cloned into the BlueScript vector and transformed into the E. coli strain XL 1 Blue. About 50,000 clones were obtained, wherein 0.5% of those were fluorescent clones, comprising a complete coding sequence of a fluorescent protein.

### Example 5

### Preparation of Directional cDNA Libraries

The method of the present invention allows synthesis of cDNA that can be effectively used for construction of directional cDNA using as a template from 100-1000 ng of total or poly(A)+RNA. In this case the structure of the cDNA synthesis primer and adaptors can be varied without changing the efficiency of the procedure.

For example, the adapter Ad8P (SEQ ID NO: 16, 3′ end residue comprises 3-phosphate group) and cDNA synthesis primer CDS-3M (SEQ ID NO: 17) comprising asymmetrical Sfi I restriction enzyme sites (A & B) can be used for cDNA synthesis. Incorporation of asymmetrical Sfi I restriction enzyme sites into cDNA allows directional cloning of the cDNA library.

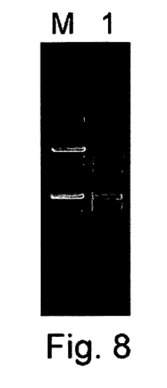

10 pmol of the CDS-3M primer was mixed with 500 ng of total RNA (Human Cerebellum) in a volume of 5 μl of sterile water and annealed to RNA by heating the mixture for 2 minutes at 70° C., followed by decreasing the temperature to 42° C. First-strand cDNA synthesis was then initiated by mixing the primer-RNA mixture with 1 μl of Power Script reverse transcriptase (Clontech) in a final volume of 10 μl, containing 50 mM Tris-HCl (pH 8.3); 75 mM KCl; 6 mM MgCl₂; 1 mM DTT; and 1 mM each of dNTP. The first-strand cDNA synthesis reaction was incubated at 42° C. for 30 min under mineral oil. After this, the template switching reaction was initiated by addition of MnCl₂ (to a final concentration of 3 mM) and 10 pmol of the Ad8P adapter to the RNA-first strand cDNA-revertase intermediates. Incubation was prolonged for 2 hours. On the reaction completion, 2 μl of the first strand cDNAs synthesized were used for PCR amplification using the Advantage 2 PCR Kit (Clontech) with the PCR primer (SEQ ID NO:10). 20 PCR cycles (95° C.-20 sec; 66° C.-20 sec; 72° C.-3 min) were performed. PCR products were revealed by gel electrophoresis on 1.2% agarose with ethidium bromide (FIG. 8). The total amount of cDNA was about 2.5 μg (25 μg/μl).

After phenol-chloroform purification and ethanol sedimentation, cDNA was treated by Sfi I restriction enzyme (Sibenyme) (1 unit onto 50-60 ng DNA). Then cDNA was purified using phenol-chloroform extraction, size fractionated by CHROMA SPIN-1000 Column (according CHROMA SPIN™ Purification Protocol, Clontech), precipitated by ethanol, and cloned into Sfi I-digested dephosphorylated λTriplEx2 vector (Clontech). Cloned cDNA was then transformed by electroporation into the E. coli strain XL 1 Blue. About 1×10⁶ independent clones were obtained.

1. A method of first-strand cDNA preparation, comprising the steps of:
(1) annealing a cDNA synthesis primer to RNA templates and synthesizing first cDNA strands to form RNA-cDNA intermediates using an enzyme which possesses reverse transcriptase activity;
(2) contacting a reaction mixture from step (1) comprising the RNA-cDNA intermediates and the enzyme which possesses reverse transcriptase activity with a deoxyribooligonucleotide adapter and Mn²⁺-ions to permit a template switching reaction, wherein said adapter comprises:
a pre-selected arbitrary nucleotide sequence at its 5′-end; and
a 3′-end terminator deoxyribonucleotide.

2. The method according to claim 1, wherein step (2) follows step (1).

3. The method according to claim 1, wherein steps (1) and (2) are performed simultaneously.

4. The method according to claim 1, wherein the enzyme which possesses reverse transcriptase activity is a MMLV-related reverse transcriptase.
